(19) United States
(12) Patent Application Publication
(10) Pub. No.: US 2023/0129987 A1
(43) Pub. Date: Apr. 27, 2023

(54) SYSTEMS AND METHODS FOR RADIOTHERAPY PLANNING

(71) Applicant: SHANGHAI UNITED IMAGING HEALTHCARE CO., LTD., Shanghai (CN)

(72) Inventors: Kang ZHANG, Shanghai (CN); Jingjie ZHOU, Shanghai (CN)

(73) Assignee: SHANGHAI UNITED IMAGING HEALTHCARE CO., LTD., Shanghai (CN)

(21) Appl. No.: 18/048,498

(22) Filed: Oct. 21, 2022

(30) Foreign Application Priority Data
Oct. 21, 2021 (CN) 202111229165.2
Oct. 26, 2021 (CN) 202111247245.0

(51) Int. Cl. A61N 5/10; G16H 20/40

(52) U.S. Cl. CPC A61N 5/10; G16H 20/40

(57) ABSTRACT

The present disclosure relates to systems and methods for radiotherapy planning. The systems may obtain a delineation of a region of interest (ROI) in an image of an object. The ROI may include at least one target region. The systems may obtain modified delineation of the ROI based on one or more modifications to the delineation of the ROI. The systems may determine a target radiotherapy plan of the object by performing a radiotherapy dose optimization on the ROI. The modifications of the delineation of the ROI and the radiotherapy dose optimization of the ROI may be performed at least partially overlap temporally.

### CROSS-REFERENCE TO RELATED APPLICATIONS

This application claims priority of Chinese Patent Application No. 202111229165.2 filed on Oct. 21, 2021 and Chinese Patent Application No. 202111247245.0 filed on Oct. 26, 2021, the contents of each of which are incorporated herein by reference.

### TECHNICAL FIELD

The present disclosure generally relates to radiotherapy (RT), and in particular, to systems and methods for radiotherapy planning.

### BACKGROUND

Radiation therapy (or referred to as radiotherapy) has been widely employed in clinical treatment for cancers and other conditions. Before the radiotherapy, a radiotherapy plan that describes how the radiotherapy is planned to be performed on the subject may be generated automatically or adaptively. The quality of the radiotherapy plan may affect the accuracy and/or efficacy of the radiotherapy. The efficiency of the radiotherapy planning may affect the efficiency of diagnosis and/or execution of the treatment. Therefore, it is desirable to provide improved systems and methods for radiotherapy planning, thereby improving the accuracy of the radiotherapy plan and/or the efficiency of radiotherapy planning.

### SUMMARY

An aspect of the present disclosure relates to a system for radiotherapy planning. The system may include at least one storage device including a set of instructions and at least one processor in communication with the at least one storage device. When executing the set of instructions, the at least one processor may be directed to cause the system to implement operations. The operations may include obtaining a delineation of a region of interest (ROI) in an image of an object, the ROI including at least one target region; obtaining modified delineation of the ROI based on one or more modifications to the delineation of the ROI; and determining a target radiotherapy plan of the object by performing a radiotherapy dose optimization on the ROI. The modifications of the delineation of the ROI and the radiotherapy dose optimization of the ROI may be performed at least partially overlap temporally.

In some embodiments, the modified delineation of the ROI may be determined by manually modifying the delineation of the ROI.

In some embodiments, the operations may further include while performing the radiotherapy dose optimization on the ROI, outputting in real time a dose distribution result and a dose volume histogram (DVH) corresponding to the radiotherapy dose being optimized.

In some embodiments, the determining the target radiotherapy plan of the object may include determining, based on the modified delineation of the ROI, whether to perform the radiotherapy dose optimization on the ROI based on an initial radiotherapy plan.

In some embodiments, the determining, based on the modified delineation of the ROI, whether to perform the radiotherapy dose optimization on the ROI based on the initial radiotherapy plan may include in response to that the modified delineation of the ROI satisfies a condition, performing the radiotherapy dose optimization on the ROI based on the initial radiotherapy plan; and in response to that the modified delineation of the ROI fails to satisfy the condition, abandoning the initial radiotherapy plan and performing the radiotherapy dose optimization on the ROI based on the modified delineation of the ROI.

In some embodiments, the operations may further include, in response to that a difference between parameters related to the modified delineation of the ROI and the delineation of the ROI is less than a threshold, determining that the modified delineation of the ROI satisfies the condition. The parameters may include at least one of a volume or a layer count of the modified delineation of the ROI.

In some embodiments, in response to that the modified delineation of the ROI satisfies the condition, the performing the radiotherapy dose optimization on the ROI based on the initial radiotherapy plan may include performing the radiotherapy dose optimization on the ROI by optimizing, using an automatic optimization algorithm, a shape and a weight of at least one segment of the initial radiotherapy plan.

In some embodiments, in response to that the modified delineation of the ROI fails to satisfy the condition, the performing the radiotherapy dose optimization on the ROI based on the modified delineation of the ROI may include performing the radiotherapy dose optimization on the ROI by optimizing, based on the modified delineation of the ROI using an automatic optimization algorithm, a fluence map related to the radiotherapy dose of the ROI.

In some embodiments, the determining the target radiotherapy plan of the object may include determining the target radiotherapy plan of the object based on the fluence map relating to the radiotherapy dose.

In some embodiments, the determining the target radiotherapy plan of the object may include determining at least one predicted delineation of the ROI by predicting a modification of the delineation of the ROI; determining at least one predicted radiotherapy plan of the object by performing, based on the at least one predicted delineation of the ROI, the radiotherapy dose optimization on the ROI; and determining the target radiotherapy plan of the object by evaluating, based on the modified delineation of the ROI, the at least one predicted radiotherapy plan.

In some embodiments, the determining the target radiotherapy plan of the object may include determining a probability density distribution of each of voxels or pixels corresponding to the ROI, the probability density distribution indicating a probability that the each voxel or pixel belongs to the ROI; determining a predicted radiotherapy plan of the object by performing, based on probability density distributions corresponding to the pixels or voxels in the ROI, the radiotherapy dose optimization on the ROI; and determining the target radiotherapy plan of the object by evaluating, based on the modified delineation, the predicted radiotherapy plan.

In some embodiments, the target radiotherapy plan may be determined online during a radiotherapy treatment session of the object that includes a delivery of the radiotherapy dose to the at least one target region of the object.

In some embodiments, the image of the object may be an identification image that is determined using a trained identification model. The trained identification model may be configured to delineate the at least one target region and the specific region in an initial image to determine the identification image. The operations may further include determining a radiotherapy dose of the specific region based on the identification image.

In some embodiments, the trained identification model may include a first sub-model and a second sub-model. The determining the identification image using the trained identification model may include determining, using the first sub-model, an intermediate identification image based on the initial image, the intermediate identification image including delineations of the at least one target region; and determining the identification image by delineating the specific region in the intermediate identification image using the second sub-model.

In some embodiments, the determining the radiotherapy dose of the specific region based on the identification image may include determining an optimization objective by performing a dose prediction based on the delineation of the specific region; obtaining a dose constraint corresponding to the specific region based on the optimization objective; and determining the radiotherapy dose of the specific region based on the dose constraint.

In some embodiments, the obtaining the dose constraint corresponding to the specific region based on the feature of the specific region may include determining, using a dose distribution prediction model, the dose constraint corresponding to the specific region.

In some embodiments, the second sub-model may be obtained by a training process including obtaining a plurality of training samples, each of the plurality of training samples including a sample image and a label image corresponding to the sample image, the sample image including delineations of at least one sample target region, the label image including a delineation of a sample specific region; and obtaining the second sub-model by training, based on the plurality of training samples, a preliminary second sub-model.

Another aspect of the present disclosure relates to a system for radiotherapy planning. The system may include at least one storage device including a set of instructions and at least one processor in communication with the at least one storage device. When executing the set of instructions, the at least one processor may be directed to cause the system to implement operations. The operations may include obtaining an initial image of an object, the initial image including at least one target region; determining an identification image using a trained identification model, the trained identification model being configured to delineate the at least one target region and a specific region in the initial image to determine the identification image; and determining a radiotherapy dose of the specific region based on the identification image.

In some embodiments, the trained identification model includes a first sub-model and a second sub-model. The determining the identification image using the trained identification model may include determining, using the first sub-model, an intermediate identification image based on the initial image, the intermediate identification image including delineations of the at least one target region; and determining the identification image by delineating the specific region in the intermediate identification image using the second sub-model.

A further aspect of the present disclosure relates to a method for radiotherapy planning. The method may be implemented on a computing device including at least one processor, at least one storage medium, and a communication platform connected to a network. The method may include obtaining a delineation of a region of interest (ROI) in an image of an object, the ROI including at least one target region; obtaining modified delineation of the ROI based on one or more modifications to the delineation of the ROI; and determining a target radiotherapy plan of the object by performing a radiotherapy dose optimization on the ROI. The modifications of the delineation of the ROI and the radiotherapy dose optimization of the ROI may be performed at least partially overlap temporally.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. The drawings are not to scale. These embodiments are non-limiting exemplary embodiments, in which like reference numerals represent similar structures throughout the several views of the drawings, and wherein:

FIG. 1 is a schematic diagram illustrating an exemplary medical system according to some embodiments of the present disclosure;

FIG. 2 is a schematic diagram illustrating exemplary hardware and/or software components of an exemplary computing device according to some embodiments of the present disclosure;

FIGS. 3A and 3B are block diagrams illustrating exemplary processing devices according to some embodiments of the present disclosure;

FIG. 4 is a flowchart illustrating an exemplary process for radiotherapy planning according to some embodiments of the present disclosure;

FIG. 5 is a schematic diagram illustrating an exemplary identification image according to some embodiments of the present disclosure;

FIGS. 6A-6B are schematic diagrams each of which illustrates an exemplary planned dose distribution in a radiotherapy plan according to some embodiments of the present disclosure;

FIG. 7 is a schematic diagram illustrating an exemplary trained identification model according to some embodiments of the present disclosure;

FIG. 8 is a flowchart illustrating an exemplary process for obtaining a trained identification model or a second sub-model according to some embodiments of the present disclosure;

FIG. 9 is a flowchart illustrating an exemplary process for radiotherapy planning according to some embodiments of the present disclosure;

FIG. 10 is a flowchart illustrating an exemplary process for radiotherapy planning according to some embodiments of the present disclosure;

FIG. 11 is a flowchart illustrating an exemplary process for radiotherapy planning according to some embodiments of the present disclosure; and

FIG. 12 is a flowchart illustrating an exemplary process for radiotherapy planning according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this disclosure, specify the presence of stated features, integers, steps, operations, elements, and/or components, but do not preclude the presence or addition of one or more other features, integers, steps, operations, elements, components, and/or groups thereof.

It will be understood that the terms “system,” “engine,” “unit,” “module,” and/or “block” used herein are one method to distinguish different components, elements, parts, sections, or assemblies of different levels in ascending order. However, the terms may be displaced by another expression if they achieve the same purpose.

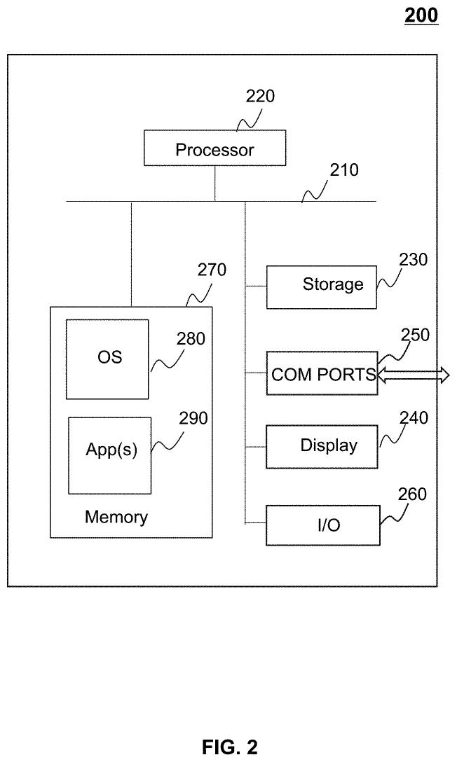

Generally, the words “module,” “unit,” or “block” used herein refer to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for performing on computing devices (e.g., processor 220 illustrated in FIG. 2) may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to performing). Such software code may be stored, partially or fully, on a storage device of the performing computing device, for performing by the computing device. Software instructions may be embedded in firmware, such as an EPROM. It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that when a unit, engine, module, or block is referred to as being “on,” “connected to,” or “coupled to” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

The flowcharts used in the present disclosure illustrate operations that systems implement according to some embodiments of the present disclosure. It is to be expressly understood, the operations of the flowcharts may be implemented not in order. Conversely, the operations may be implemented in inverted order, or simultaneously. Moreover, one or more other operations may be added to the flowcharts. One or more operations may be removed from the flowcharts.

The term “image” in the present disclosure is used to collectively refer to imaging data (e.g., scan data, projection data) and/or images of various forms, including a two-dimensional (2D) image, a three-dimensional (3D) image, a four-dimensional (4D), etc. The term “pixel” and “voxel” in the present disclosure are used interchangeably to refer to an element of an image. The term “region,” “location,” and “area” in the present disclosure may refer to a location of an anatomical structure shown in the image or an actual location of the anatomical structure existing in or on a target subject's body, since the image may indicate the actual location of a certain anatomical structure existing in or on the target subject's body.

Generally, in automatic or adaptive radiotherapy planning, a delineation of a region of interest (ROI) (e.g., at least one target region, at least one organ at risk (OAR), and/or a specific region) needs to be reviewed and modified by a user (e.g., a radiation physicist or doctor), and then the automated planning system generates a radiotherapy plan based on the modified delineation of the ROI. It is time-consuming for the user to review and modify the delineation of the ROI, and the existing automatic or adaptive radiotherapy planning can only be started after the user completes the delineation modification. To improve the efficiency of automatic or adaptive radiotherapy planning, an aspect of the present disclosure provides systems and methods for radiotherapy planning. In some embodiments, the systems may be configured to obtain the delineation of ROI in an image of an object. The systems may be configured to obtain modified delineation of the ROI based on one or more modifications to the delineation of the ROI. The systems may determine a target radiotherapy plan of the object by performing a radiotherapy dose optimization on the ROI. In some embodiments, the modifications of the delineation of the ROI and the radiotherapy dose optimization of the ROI are performed in parallel (e.g., at least partially overlapping temporally), so that the time for automatic or adaptive radiotherapy planning is reduced, thereby improving the efficiency of the automatic or adaptive radiotherapy planning.

Generally, an automated planning system obtains a radiotherapy plan according to either one of two manners. According to the first manner, a user, e.g., an experienced radiation physicist or doctor, manually delineates a specific region (e.g., a region in a vicinity of a lesion); the automated planning system sets a radiotherapy dose of the specific region. The automated planning system generates a radiotherapy plan based on the delineated specific region and the radiotherapy dose of the specific region. The radiotherapy planning so performed may be inefficient, cause a substantial workload on the user (e.g., an experienced radiation physicist or doctor), and/or rely on expertise and/or experience of the user. According to the second manner, a three-dimensional predicted dose distribution in a patient, or a portion thereof, is obtained in a certain way. The automated planning system generates the radiotherapy plan based on the three-dimensional predicted dose distribution using an optimization algorithm. The three-dimensional dose distribution may be inaccurate and cannot accurately reflect the radiotherapy dose of the specific region. Even if the three-dimensional dose distribution is sufficiently accurate, it may be difficult to accurately identify a location of the specific region and/or give appropriate weight to control the radiotherapy dose of the specific region according to the optimization algorithm.

Another aspect of the present disclosure provides systems and methods for radiotherapy planning. In some embodiments, the systems may be configured to obtain an initial image (e.g., a CT image) of an object (e.g., a patient). The image may include at least one target region (e.g., a region to be treated in radiotherapy) and at least one organ at risk (OAR) (e.g., an organ that needs to be protected from over-radiation). The systems may determine an identification image using a trained identification model (e.g., a deep learning model). The trained identification model may be configured to delineate the at least one target region, the at least one OAR, and a specific region in the image to determine the identification image. Further, the systems may determine a radiotherapy dose of the specific region based on the identification image.

According to some embodiments of the present disclosure, by using the trained identification model, the identification and/or delineation of the specific region is automated, thereby improving the efficiency and/or accuracy of the identification and/or delineation of various regions (including, e.g., at least one target region, an OAR, a specific region) and the treatment planning on the basis thereof, reducing the workload and/or cross-user variation involved in the treatment planning. Further, the systems and methods of some embodiments of the present disclosure may effectively and accurately control the delivery of a treatment including the radiotherapy doses toward various regions (including, e.g., at least one target region, an OAR, a specific region), thereby improving the efficacy of the treatment.

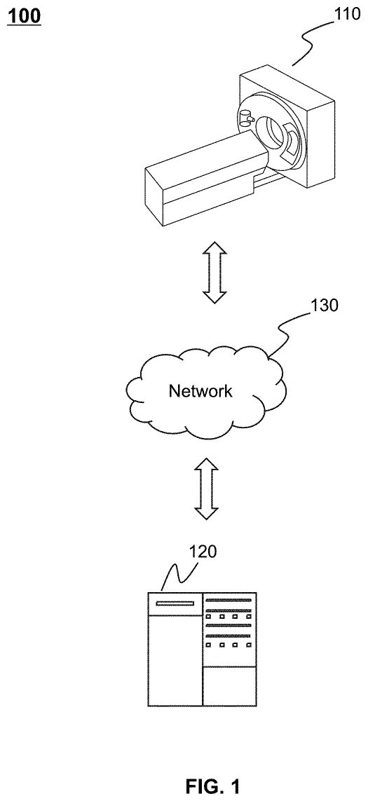

FIG. 1 is a schematic diagram illustrating an exemplary medical system according to some embodiments of the present disclosure. As illustrated, the medical system 100 may include a radiation device 110, a processing device 120, and a network 130. The components of the medical system 100 may be connected in one or more of various ways. Mere by way of example, as illustrated in FIG. 1, the radiation device 110 may be connected to the processing device 120 through the network 130. As another example, the radiation device 110 may be connected to the processing device 120 directly.

The radiation device 110 may be configured to perform a medical procedure on an object (or a portion thereof) by emitting radiation beams (e.g., x-ray, gamma-ray) toward the object (or the portion thereof). In the present disclosure, the object may include a biological object and/or a non-biological object. The biological object may be a human being, an animal, a plant, or a specific portion, organ, and/or tissue thereof. For example, the object may include a head, a neck, a thorax, a heart, a stomach, a blood vessel, a soft tissue, a tumor, a nodule, or the like, or any combination thereof. In some embodiments, the object may be a man-made composition of organic and/or inorganic matters that are with or without life.

In the present disclosure, a representation of an object (e.g., a patient, a subject, or a portion thereof) in an image may be referred to as “object” for brevity. For instance, a representation of an organ or tissue (e.g., a heart, a liver, a lung) in an image may be referred to as an organ or tissue for brevity. Further, an image including a representation of an object may be referred to as an image of an object or an image including an object for brevity. Still further, an operation performed on a representation of an object in an image may be referred to as an operation performed on an object for brevity. For instance, a segmentation of a portion of an image including a representation of an organ or tissue from the image may be referred to as segmentation of an organ or tissue for brevity.

In some embodiments, the radiation device 110 may be or include an imaging device. The imaging device may be configured to acquire imaging data relating to the object. For example, the imaging device may scan the object or a portion thereof that is located within its detection region and generate imaging data relating to the object or the portion thereof. The imaging data relating to the object may include an image, projection data, or a combination thereof. In some embodiments, the imaging data may include two-dimensional (2D) imaging data (e.g., a slice image), three-dimensional (3D) imaging data, four-dimensional (4D) imaging data (a series of 3D images over time), or the like, or any combination thereof.

In some embodiments, the radiation device 110 may be or include a treatment device (e.g., an RT device). The treatment device may be configured to deliver a radiotherapy treatment to the object. For example, the treatment device may deliver one or more radiation beams to a treatment region (e.g., a tumor) of an object for causing an alleviation of the object's symptom.

The processing device 120 may control the radiation device 110 and process data and/or information obtained from the radiation device 110. For example, the processing device 120 may obtain an initial image of an object from the imaging device of the radiation device 110 and delineate at least one target region, at least one OAR, and a specific region in the image using a trained identification model. Further, the processing device 120 may obtain a modified delineation of a region of interest (ROI) in the identification image. The ROI may include at least one of the at least one target region, the at least one OAR, or the specific region. The processing device 120 may determine a target radiotherapy plan by performing a radiotherapy dose optimization on the ROI. The processing device 120 may transmit the target radiotherapy plan to the treatment device of the radiation device 110 to deliver a radiotherapy treatment to the object based on the target radiotherapy plan. In some embodiments, the processing device 120 may include a central processing unit (CPU), a digital signal processor (DSP), a system on a chip (SoC), a microcontroller unit (MCU), or the like, or any combination thereof. In some embodiments, the processing device 120 may include a mobile device, a computer (e.g., a tablet computer, a laptop computer), a wearable device, a user console, a single server, or a server group, etc. In some embodiments, the processing device 120 or a portion of the processing device 120 may be integrated into the radiation device 110. In some embodiments, the processing device 120 may be implemented by a computing device 200 including one or more components as described in FIG. 2.

The network 130 may include any suitable network that can facilitate the exchange of information and/or data for the medical system 100. In some embodiments, one or more components (e.g., the radiation device 110, the processing device 120) of the medical system 100 may communicate information and/or data with one or more other components of the medical system 100 via the network 130. For example, the processing device 120 may obtain imaging data from the radiation device 110 via the network 130. In some embodiments, one or more components (e.g., the radiation device 110, the processing device 120) of the medical system 100 may communicate information and/or data with one or more external resources such as an external database of a third party, etc.

It should be noted that the above description of the medical system 100 is merely provided for the purposes of illustration, and not intended to limit the scope of the present disclosure. For persons having ordinary skills in the art, multiple variations and modifications may be made under the teachings of the present disclosure. For example, the medical system 100 may include one or more additional components (e.g., a storage device) and/or one or more components of the medical system 100 described above may be omitted. Additionally or alternatively, two or more components of the medical system 100 may be integrated into a single component. A component of the medical system 100 may be implemented on two or more sub-components.

FIG. 2 is a schematic diagram illustrating exemplary hardware and/or software components of an exemplary computing device according to some embodiments of the present disclosure. In some embodiments, the processing device 120 may be implemented on the computing device 200. In some embodiments, the computing device 200 may be a terminal. As illustrated in FIG. 2, the computing device 200 may include a data bus 210, a processor 220, a storage 230, a display 240, a communication port 250, an input/output (I/O) 260, and a memory 270.

The data bus 210 may be configured to implement data communications among components of the computing device 200. In some embodiments, hardware in the computing device 200 may transmit data via the data bus 210. For example, the processor 220 may send data to a storage or other hardware such as the I/O 260 via the data bus 210.

The processor 220 may execute computer instructions (program code) and perform functions of the processing device 120 in accordance with techniques described herein. Merely for illustration purposes, only one processor is described in the computing device 200. However, it should be noted that the computing device 200 in the present disclosure may also include multiple processors, and thus operations of a method that are performed by one processor as described in the present disclosure may also be jointly or separately performed by the multiple processors.

The storage 230 may store data/information obtained from the radiation device 110 of the medical system 100. In some embodiments, the storage 230 may store one or more programs and/or instructions to perform exemplary methods described in the present disclosure. For example, the storage 230 may store a program for the processing device 120 for determining an identification image using a trained identification model.

The display 240 may include a liquid crystal display screen or an electronic ink display screen. An input device of the computing device 200 may be a touch layer covered on the display 240, or a button, a trackball, or a touchpad set on a shell of the computing device 200, or an external keyboard, trackpad, or mouse connected to the computing device 200.

The communication port 250 may be connected to a network (e.g., the network 130) to facilitate data communications. The communication port 250 may establish connections between the processing device 120 and the radiation device 110 of the medical system 100. The connection may be a wired connection, a wireless connection, or a combination of both that enables data transmission and reception.

The I/O 260 may input or output signals, data, or information. In some embodiments, the I/O 260 may enable user interaction with the processing device 120. In some embodiments, the I/O 260 may include an input device and an output device. Merely by way of example, a user (e.g., an operator) may input parameters needed for the operation of the radiation device 110.

A mobile operating system 280 (e.g., iOS, Android, Windows Phone) and one or more applications 290 may be loaded into the memory 270 from the storage 230 in order to be executed by the processor 220.

t should be noted that the above description of the computing device 200 is merely provided for the purposes of illustration, and not intended to limit the scope of the present disclosure. For persons having ordinary skills in the art, multiple variations and modifications may be made under the teachings of the present disclosure.

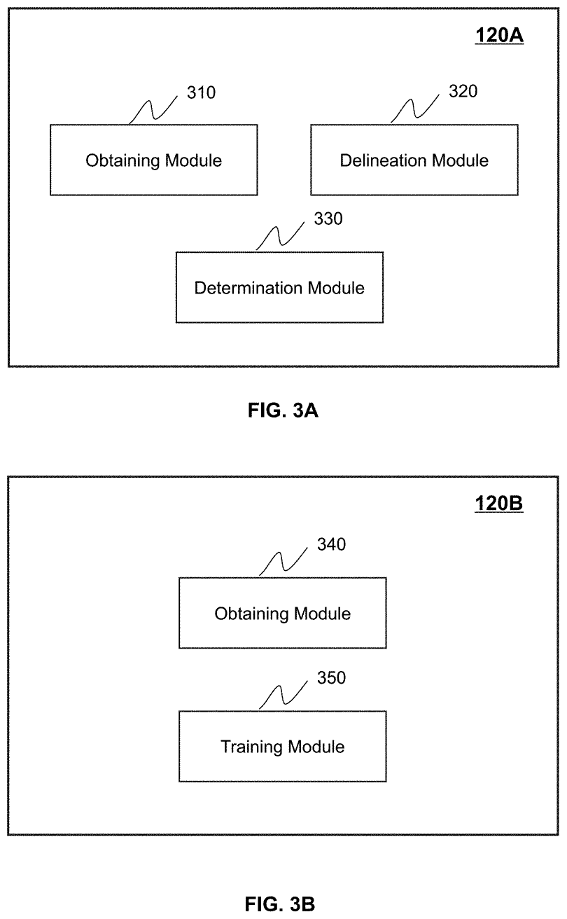

FIGS. 3A and 3B are block diagrams illustrating exemplary processing devices 120A and 120B according to some embodiments of the present disclosure. The processing devices 120A and 120B may be exemplary processing devices 120 as described in connection with FIG. 1. In some embodiments, the processing device 120A may be configured to apply a trained identification model for delineating at least one target region, at least one OAR, and a specific region in an initial image. The processing device 120B may be configured to obtain a plurality of training samples and/or determine one or more models (e.g., a trained identification model, a second sub-model of the trained identification model) using the training samples. In some embodiments, the processing devices 120A and 120B may be respectively implemented on a processing unit (e.g., a processor 220 illustrated in FIG. 2). Alternatively, the processing devices 120A and 120B may be implemented on a same computing unit.

As shown in FIG. 3A, the processing device 120A may include an obtaining module 310, a delineation module 320, and a determination module 330.

The obtaining module 310 may be configured to obtain an initial image of an object. More descriptions regarding the obtaining of the initial image may be found elsewhere in the present disclosure. See, e.g., operation 410 and relevant descriptions thereof.

The delineation module 320 may be configured to determine an identification image using a trained identification model. More descriptions regarding the determining of the identification image may be found elsewhere in the present disclosure. See, e.g., operation 420 and relevant descriptions thereof.

The determination module 330 may be configured to determine a radiotherapy plan of the object based on the identification image. More descriptions regarding the determining of the radiotherapy plan of the object may be found elsewhere in the present disclosure. See, e.g., operation 430 and relevant descriptions thereof.

The delineation module 320 may be further configured to obtain a delineation of a region of interest (ROI) in an image of an object. More descriptions regarding the obtaining of the delineation of the ROI may be found elsewhere in the present disclosure. See, e.g., operation 910 and relevant descriptions thereof.

The delineation module 320 may be further configured to obtain a modified delineation of the ROI based on one or more modifications to the delineation of the ROI. More descriptions regarding the obtaining of the modified delineation of the ROI may be found elsewhere in the present disclosure. See, e.g., operation 920 and relevant descriptions thereof.

The determination module 330 may be further configured to determine a target radiotherapy plan of the object by performing a radiotherapy dose optimization on the ROI. More descriptions regarding the determining of the target radiotherapy plan of the object may be found elsewhere in the present disclosure. See, e.g., operation 930 and relevant descriptions thereof.

As shown in FIG. 3B, the processing device 120B may include an obtaining module 340 and a training module 350.

The obtaining module 340 may be configured to obtain a plurality of training samples. More descriptions regarding the obtaining of the plurality of training samples may be found elsewhere in the present disclosure. See, e.g., operation 810 and relevant descriptions thereof.

The training module 350 may be configured to obtain the trained identification model or the second sub-model based on the plurality of training samples. More descriptions regarding the obtaining of the trained identification model or the second sub-model may be found elsewhere in the present disclosure. See, e.g., operation 820 and relevant descriptions thereof.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, the processing device 120A and/or the processing device 1206 may share two or more of the modules, and any one of the modules may be divided into two or more units. For instance, the processing devices 120A and 1206 may share a same obtaining module; that is, the obtaining module 310 and the obtaining module 340 are a same module. In some embodiments, the processing device 120A and/or the processing device 1206 may include one or more additional modules, such as a storage module (not shown) for storing data. In some embodiments, the processing device 120A and the processing device 1206 may be integrated into one processing device 120.

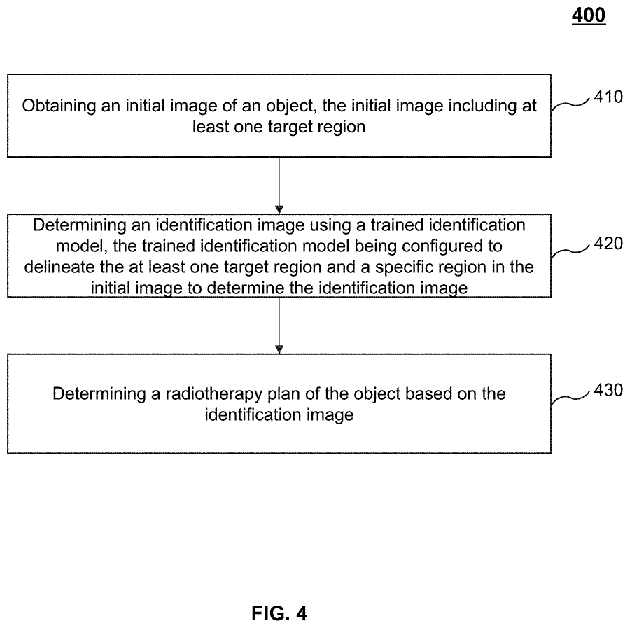

FIG. 4 is a flowchart illustrating an exemplary process for radiotherapy planning according to some embodiments of the present disclosure. In some embodiments, process 400 may be executed by the medical system 100. For example, the process 400 may be implemented as a set of instructions (e.g., an application) stored in a storage device (e.g., the storage 230). In some embodiments, the processing device 120A (e.g., the processor 220 of the computing device 200 and/or one or more modules illustrated in FIG. 3A) may execute the set of instructions and may accordingly be directed to perform the process 400.

In 410, the processing device 120A (e.g., the obtaining module 310 illustrated in FIG. 3A, the processor 220 illustrated in FIG. 2) may obtain an initial image of an object. The object may include a patient, or a portion thereof. For instance, the object may include at least one target region, an organ at risk (OAR), etc., of a patient.

In some embodiments, the initial image may include a CT image, a cone beam CT (CBCT) image, a fan beam CT (FBCT) image, an MR image, a PET-CT image, a PET-MR image, a SPECT-CT Image, a SPECT-MR Image, or the like, or a combination thereof. In some embodiments, the initial image may include at least one target region (also referred to as “target” or “target volume”) and at least one organ at risk (OAR) of a patient. As used herein, a representation of an object (e.g., a patient, or a portion thereof) in an image or image data may be referred to as the object for brevity. For instance, a representation of an organ or tissue of a patient (e.g., the heart, the liver, a lung, etc., of the patient) in an image or image data may be referred to as the organ or tissue for brevity. An image or image data including a representation of an object may be referred to as an image or image data of the object or an image or image data including the object for brevity. As used herein, an operation on a representation of an object in an image or image data may be referred to as an operation on the object for brevity. For instance, a segmentation of a portion of an image or image data including a representation of an organ or tissue of a patient (e.g., the heart, the liver, a lung, etc., of the patient) from the image or image data may be referred to as a segmentation of the organ or tissue for brevity. As used herein, at least one target region of a patient refers to a region of the patient that includes a volume to be treated using radiation. The at least one target region may include at least part of malignant tissue (e.g., a tumor, a cancer-ridden organ, or a non-cancerous target of radiation therapy). For example, the at least one target region may be a tumor, an organ with a tumor, a tissue with a tumor, or any combination thereof, that needs to be treated by radiation. As another example, when the initial image is an abdomen image, the at least one target region may include the liver. As a further example, when the initial image is a neck image, the at least one target region may include a lymph node in the neck. The OAR may include an organ and/or tissue that is close to the at least one target region and not intended to be subjected to radiation, but under the risk of radiation damage due to its proximity to the at least one target region.

In some embodiments, the processing device 120A may direct the imaging device of the radiation device 110 to perform a scan (e.g., a CT scan) on the object and determine the initial image based on scanning data obtained from the imaging device. In some embodiments, the initial image may be previously determined and stored in a storage device (e.g., the storage 230, an external storage device) or an external system (e.g., a picture archiving and communication system (PACS)). The processing device 120A may retrieve the initial image from the storage device or the external system directly or via a network (e.g., the network 130).

In 420, the processing device 120A (e.g., the delineation module 320 illustrated in FIG. 3A, the processor 220 illustrated in FIG. 2) may determine an identification image using a trained identification model.

As used herein, a trained identification model refers to a model (e.g., a machine learning model) or an algorithm configured for delineating a region in an image based on its input. As used herein, a delineation of a region in an image refers to determining an outline or boundary of the region in the image. In some embodiments, the outline or boundary of a region in an image may be presented as a line with a certain color for display. In some embodiments, the trained identification model may be configured to determine the identification image by delineating the at least one target region, the at least one OAR, and a specific region in the initial image. As used herein, a specific region refers to a region that is in a vicinity of and outside where the at least one target region and/or the at least one OAR are located. The specific region may include a region with a special clinical need, a region of concern, a region where the radiotherapy dose needs to be controlled (e.g., to be below a radiotherapy dose threshold), a region that has an impact on the dose control of the at least one target region or the at least one OAR, or the like, or a combination thereof.

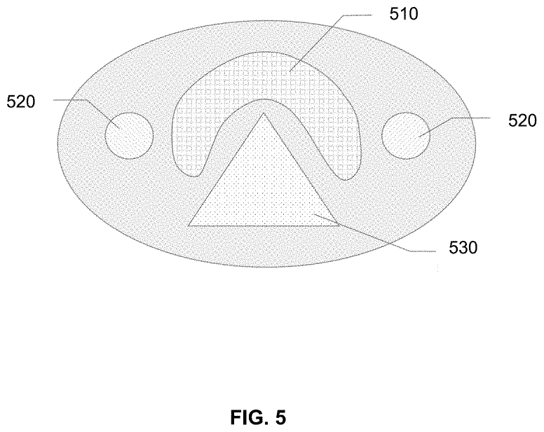

In some embodiments, the processing device 120A may determine the identification image by inputting the initial image into the trained identification model. FIG. 5 is a schematic diagram illustrating an exemplary identification image according to some embodiments of the present disclosure. As illustrated in FIG. 5, at least one target region 510, two OARs 520, and a specific region 530 are delineated.

In some embodiments, an output of the identification model may include features of the at least one target region, the at least one OAR, and/or the specific region. The features of the at least one target region, the at least one OAR, and/or the specific region may include a size, a location, and/or a shape of the at least one target region, the at least one OAR, and/or the specific region. In some embodiments, the output of the identification model does not include such features of the at least one target region, the at least one OAR, and/or the specific region, and the processing device 120A may determine the features of the at least one target region, the at least one OAR, and/or the specific region based on the identification image.

In some embodiments, the trained identification model may include a trained machine learning model, for example, a trained neural network model. Exemplary neural network models may include a deep neural network (DNN) model, a convolutional neural network (CNN) model, a recurrent neural network (RNN) model, a feature pyramid network (FPN) model, or the like, or any combination thereof. In some embodiments, the processing device 120A may obtain the trained identification model from one or more components of the medical system 100 or an external source via a network (e.g., the network 130). For example, the trained identification model may be previously trained by a computing device (e.g., the processing device 120B), and stored in a storage device (e.g., the storage 230) of the medical system 100. The processing device 120A may access the storage device and retrieve the trained identification model from the storage device. In some embodiments, the trained identification model may be generated according to a machine learning algorithm (e.g., an artificial neural network algorithm, a deep learning algorithm, etc.). In some embodiments, the trained identification model may be generated by a computing device (e.g., the processing device 120B) by performing a process (e.g., process 800) for generating a trained identification model disclosed herein.

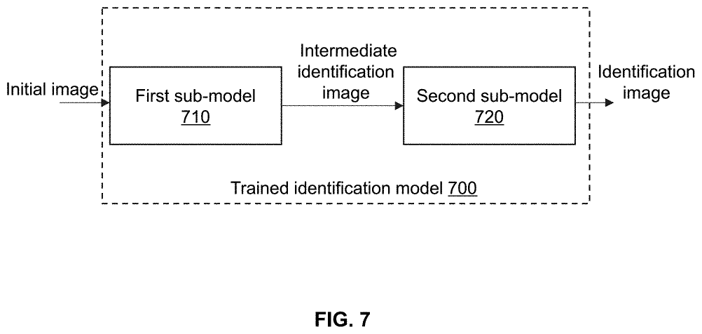

In some embodiments, the trained identification model may include a first sub-model (also referred to as “segmentation sub-model”) and a second sub-model (also referred to as “identification sub-model”). The first sub-model may be configured to delineate the at least one target region and the at least one OAR in the initial image to determine an intermediate identification image (also referred to as “a segmentation image”). The second sub-model may be configured to delineate the specific region in the intermediate identification image to determine the identification image. In some embodiments, the first sub-model may be an existing trained model, and the second sub-model may be generated by a computing device (e.g., the processing device 1206) by performing a process (e.g., process 800). More descriptions regarding the trained identification model may be found elsewhere in the present disclosure. See, e.g., FIG. 7 and the descriptions thereof.

According to some embodiments of the present disclosure, by using the trained identification model, the at least one target region, the at least one OAR, and the specific region may be delineated automatically, thereby improving the efficiency and/or accuracy of the delineation/identification of the at least one target region, the at least one OAR, and the specific region and/or reducing the workload of a user (e.g., a radiation physicist, a radiotherapy specialist, or a doctor) and/or cross-user variation.

In 430, the processing device 120A (e.g., the determination module 330 illustrated in FIG. 3A, the processor 220 illustrated in FIG. 2) may determine a radiotherapy plan of the object based on the identification image.

In some embodiments, the processing device 120A may determine radiotherapy doses of the at least one target region, the at least one OAR, and/or the specific region based on the identification image. As used herein, a radiotherapy dose of a region (e.g., the at least one target region, the at least one OAR, the specific region) refers to a dose of radiation that is planned to be delivered to the region during radiotherapy.

The processing device 120A may obtain a dose constraint corresponding to the region based on one or more features of the region including the at least one target region, the at least one OAR, the specific region, or the like, or a combination thereof. As used herein, the dose constraint corresponding to the region indicates that a radiotherapy dose delivered during radiotherapy (or a portion thereof, e.g., a treatment session of radiotherapy including a series of treatment sessions) to the region, or a portion thereof, needs to satisfy specific constrain(s) (e.g., lower than a threshold value). The dose constraint may include, for example, a maximum total radiation dose delivered to the region (or a portion thereof) in a treatment session or in the entire radiotherapy, a maximum radiation dose delivered to the region (or a portion thereof) at any time point during the treatment session or in the entire radiotherapy, a maximum radiation dose delivered to the object (or a portion thereof) in a treatment session or in the entire radiotherapy, a dose distribution in the region (or a portion thereof) in the treatment session or in the entire radiotherapy, or the like, or any combination thereof. In some embodiments, different regions may correspond to different dose constraints. A corresponding relationship between the feature(s) of a region (e.g., the at least one target region, the at least one OAR, the specific region) and the dose constraint of the region may be previously determined and stored in a storage device (e.g., the storage 230, an external storage device). The processing device 120A may obtain the dose constraint of the region from the storage device based on the feature(s) of the region. In some embodiments, the dose constraint of a region (e.g., the at least one target region, the at least one OAR, the specific region) may be input by a user (e.g., a radiation physicist or doctor).

In some embodiments, the processing device 120A may determine the dose constraint corresponding to a region (e.g., the at least one target region, the at least one OAR, the specific region) using a dose distribution prediction model (e.g., a machine learning model, a neural network model). For instance, the processing device 120A may determine a dose distribution by inputting the identification image into the dose distribution prediction model. The processing device 120A may determine, based on the dose distribution of the region, the dose constraint corresponding to the region. For example, the processing device 120A may designate the dose distribution of the region as the dose constraint corresponding to the region. As another example, the processing device 120A may assess the dose constraint corresponding to the region based on a minimum value, a maximum value, an average value, a median value, or another characteristic value of the dose distribution of the region. In some embodiments, the dose distribution prediction model may be previously trained by a computing device (e.g., the processing device 120B). For example, the processing device 1206 may train the dose distribution prediction model based on sample images and corresponding sample dose distributions in the sample images.

In some embodiments, the processing device 120A may determine an optimization objective by performing a dose prediction based on the delineation of a region (e.g., the at least one target region, the at least one OAR, the specific region). The optimization objective may include dose information associated with the region in a treatment session or in the entire radiotherapy. The processing device 120A may obtain the dose constraint corresponding to the region based on the optimization objective. Merely by way of example, the optimization objective may include the dose constraint corresponding to the region, and the processing device 120A may obtain the dose constraint corresponding to the region from the optimization objective.

Further, the processing device 120A may determine the radiotherapy dose of the region (e.g., the at least one target region, the at least one OAR, the specific region) based on the dose constraint. In some embodiments, the dose constraint may be used to construct a function of an optimization model. The function of the optimization model may assess a degree of deviation between the radiotherapy dose of the region and the dose constraint. For instance, the optimization model may be configured to reduce the degree of deviation between the radiotherapy dose of the region and the dose constraint. The processing device 120A may determine the radiotherapy dose of the region using the optimization model.

In some embodiments, the radiotherapy plan of the object may include the radiotherapy dose of the specific region. The radiotherapy plan may describe how a radiation treatment is to be performed on the object. Merely by way of example, the radiation treatment may be delivered to the object during several treatment sessions, spread over a treatment period of multiple days (e.g., 2 to 5 weeks). The radiotherapy plan may include information including, e.g., how one or more beams are delivered to at least one target region of the object during each treatment session over the course of treatment. For example, the radiotherapy plan may provide a total dose (e.g., 0.1 Gy, 10 Gy, 50 Gy, 100 Gy, etc.) and a dose distribution in the object during each treatment session.

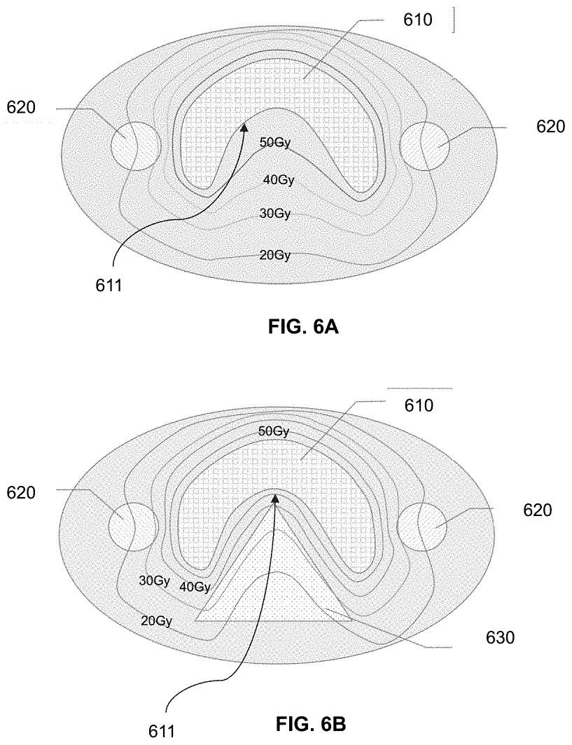

FIGS. 6A-6B are schematic diagrams each of which illustrates an exemplary dose distribution in a radiotherapy plan according to some embodiments of the present disclosure. As illustrated in FIG. 6A, at least one target region 610 and two OAR 620 are delineated. FIG. 6A illustrates a dose distribution of a radiotherapy plan that is generated without controlling the radiotherapy dose of a specific region 630. It can be seen from FIG. 6A that dose lines (especially dose lines of 20 Gy and 30 Gy) in a vicinity of a concave side 611 of the at least one target region 610 do not conform to a shape of the concave side 611 of the at least one target region 610. As used herein, a dose line is a line along which the radiation dose is constant. That is, the radiotherapy doses in the vicinity of the concave side 611 of the at least one target region 610 fall slowly, indicating that the vicinity of the concave side 611 of the at least one target region 610 receives a high radiotherapy dose.

As illustrated in FIG. 6B, the at least one target region 610, the two OAR 620, and the specific region 630 are delineated. FIG. 6B illustrates a dose distribution of a radiotherapy plan that is generated by controlling the radiotherapy dose of the specific region 630. In FIG. 6B, the dose line in a vicinity of the concave side 611 of the at least one target region 610 (e.g., in the specific region 630) conforms to the shape of the concave side 611 of the at least one target region 610. That is, the radiotherapy doses in the specific region 630 drop fast, thereby avoiding the specific region 630 from receiving a high radiotherapy dose, and/or improving the efficacy (or reducing the side effect) of the corresponding radiotherapy plan.

It should be noted that the above description of the process 400 is merely provided for the purposes of illustration, and not intended to limit the scope of the present disclosure. For persons having ordinary skills in the art, multiple variations or modifications may be made under the teachings of the present disclosure. However, those variations and modifications do not depart from the scope of the present disclosure. For example, when there are multiple specific regions in an initial image that needs to be delineated, each of the multiple specific regions is delineated using a separate trained identification model or all specific regions are delineated using a same trained identification model.

FIG. 7 is a schematic diagram illustrating an exemplary trained identification model according to some embodiments of the present disclosure.

As illustrated in FIG. 7, a trained identification model 700 includes a first sub-model 710 and a second sub-model 720. The first sub-model 710 may be configured to delineate at least one target region and at least one OAR in an initial image to determine an intermediate identification image. For example, an initial image of an object may be input into the first sub-model 710. The first sub-model 710 may generate an intermediate identification image. In the intermediate identification image, the at least one target region and the at least one OAR may be delineated.

In some embodiments, the first sub-model 710 may determine one or more features (e.g., a size, a location, or a shape) of the at least one target region and the at least one OAR in the input image. The at least one target region and the at least one OAR may be manually delineated by a user (e.g., a radiation physicist or doctor) based on the determined features of the at least one target region and the at least one OAR. The initial image that has been manually delineated may be determined as the intermediate identification image. In some embodiments, the first sub-model 710 may include a trained machine learning model, for example, a trained neural network model.

The second sub-model 720 may be configured to delineate a specific region in the intermediate identification image to determine an identification image. For example, the intermediate identification image determined by the first sub-model 710 may be input into the second sub-model 720. The second sub-model 720 may generate an identification image. In the identification image, the specific region may be delineated, in addition to the at least one target region and the at least one OAR that are determined using the first sub-model 710 or manually delineated. In some embodiments, the second sub-model 720 may determine one or more features (e.g., a size, a location, or a shape) of the specific region. In some embodiments, the second sub-model 720 may include a trained machine learning model, for example, a trained neural network model.

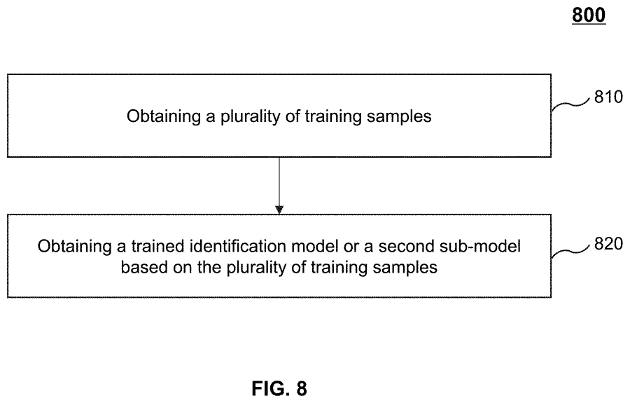

FIG. 8 is a flowchart illustrating an exemplary process for obtaining a trained identification model or a second sub-model according to some embodiments of the present disclosure. In some embodiments, process 800 may be executed by the medical system 100. For example, the process 800 may be implemented as a set of instructions (e.g., an application) stored in a storage device (e.g., the storage 230). In some embodiments, the processing device 120B (e.g., the processor 220 of the computing device 200 and/or one or more modules illustrated in FIG. 3B) may execute the set of instructions and may accordingly be directed to perform the process 800. In some embodiments, one or more operations of the process 800 may be performed to achieve at least part of operation 420 as described in connection with FIG. 4. In some embodiments, the process 800 may be performed by another device or system other than the medical system 100, e.g., a device or system of a vendor of a manufacturer that provides and/or maintains such a trained identification model or second sub-model. For illustration purposes, the following descriptions are described with reference to the implementation of the process 800 by the processing device 120B, and not intended to limit the scope of the present disclosure.

In 810, the processing device 120B (e.g., the obtaining module 340 illustrated in FIG. 3B, the processor 220 illustrated in FIG. 2) may obtain a plurality of training samples.

The plurality of training samples may include a plurality of first training samples and a plurality of second training samples. The plurality of first training samples may be used to determine the trained identification model that includes a first sub-model and a second sub-model. Each of the plurality of first training samples may include a first sample image and a first label image corresponding to the first sample image. The first sample image may be an image that includes at least one sample target region and at least one sample OAR, and the at least one sample target region and the at least one sample OAR are not delineated in the first sample image. The first label image may include delineations of the at least one sample target region, the at least one sample OAR, and the sample specific region. In some embodiments, the first label image may be obtained by manually delineating, in the first sample image, the at least one sample target region, the at least one sample OAR, and the sample specific region.

The plurality of second training samples may be used to determine the second sub-model. Each of the plurality of second training samples may include a second sample image and a second label image corresponding to the second sample image. The second sample image may include delineations of at least one sample target region and at least one sample OAR. In some embodiments, the second sample image may be obtained by manually delineating, in the first sample image, the at least one sample target region and the at least one sample OAR. The second label image may include a delineation of a sample specific region. In some embodiments, the second label image may be obtained by manually delineating, in the second sample image, the sample specific region.

In some embodiments, a training sample (e.g., a first training sample, a second training sample) may be previously generated and stored in a storage device (e.g., the storage 230, or an external database). The processing device 120B may retrieve the training sample directly or via a network (e.g., the network 130) from the storage device. In some embodiments, at least a portion of the training samples may be generated by the processing device 1206. Merely by way of example, the processing device 1206 may direct the imaging device of the radiation device 110 to perform a scan (e.g., a CT scan) on a sample object (e.g., a patient) and determine the first sample image based on scanning data obtained from the imaging device.

In 820, the processing device 120B (e.g., the training module 350 illustrated in FIG. 3B, the processor 220 illustrated in FIG. 2) may obtain the trained identification model or the second sub-model based on the plurality of training samples.

The processing device 120B may determine the trained identification model by training, based on the plurality of first training samples, a first preliminary model. The processing device 120B may determine the second sub-model by training, based on the plurality of second training samples, a second preliminary model. In some embodiments, the preliminary model (e.g., the first preliminary model, the second preliminary model) may be of any type of model including, for example, a machine learning model (e.g., a neural network model). In some embodiments, the preliminary model (e.g., the first preliminary model, the second preliminary model) may include one or more model parameters having one or more initial values before model training. The training of the preliminary model (e.g., the first preliminary model, the second preliminary model) may include one or more iterations. For illustration purposes, the following descriptions are described with reference to a current iteration. When obtaining the trained identification model, in the current iteration, the processing device 120B may input the first sample image of a first training sample into the first preliminary model (or an intermediate first model obtained in a prior iteration (e.g., the immediately prior iteration)) in the current iteration to obtain a first predicted image. The processing device 120B may determine a value of a first loss function based on the first predicted image and the first label image corresponding to the first sample image. The first loss function may be used to measure a difference between the first predicted image and the first label image. When obtaining the second sub-model, in the current iteration, the processing device 120B may input the second sample image of a second training sample into the second preliminary model (or an intermediate second model obtained in a prior iteration (e.g., the immediately prior iteration)) in the current iteration to obtain a second predicted image. The processing device 120B may determine a value of a second loss function based on the second predicted image and the second label image corresponding to the second sample image. The second loss function may be used to measure a difference between the second predicted image and the second label image.

Further, the processing device 120B may determine whether a termination condition is satisfied in the current iteration based on the value of a loss function (e.g., the first loss function, the second loss function). Exemplary termination conditions may include that the value of the loss function obtained in the current iteration is less than a predetermined threshold, that a certain count of iterations is performed, that the loss function converges such that the differences of the values of the loss function obtained in consecutive iterations are within a threshold, or the like, or any combination thereof. In response to a determination that the termination condition is satisfied in the current iteration, the processing device 1206 may designate the preliminary model (e.g., the first preliminary model, the second preliminary model) in the current iteration (or the intermediate machine learning model) as a trained model (e.g., the trained identification model, the second sub-model). Alternatively or additionally, the processing device 1206 may further store the trained model in a storage device (e.g., the storage 230) of the medical system 100 and/or output the trained model for further use (e.g., in process 400).

If the termination condition is not satisfied in the current iteration, the processing device 120B may update the preliminary (or intermediate) model (e.g., the first preliminary model, the second preliminary model) in the current iteration and proceed to a next iteration. For example, the processing device 1206 may update the value(s) of the model parameter(s) of the preliminary (or intermediate) model based on the value of the loss function (e.g., the first loss function, the second loss function) according to, for example, a backpropagation algorithm. The processing device 120B may perform the next iteration until the termination condition is satisfied. After the termination condition is satisfied in a certain iteration, the preliminary model in the certain iteration may be designated as the trained model.

It should be noted that the above descriptions regarding the process 800 are merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, the order of the process 800 and/or the process 800 may not be intended to be limiting. For example, the trained identification model and/or the second sub-model may be previously generated and stored in a storage device (e.g., the storage 230, or an external database), and the processing device 120B may retrieve the trained identification model and/or the second sub-model directly from the storage device for further use (e.g., in process 400). As another example, the processing device 120B may divide the plurality of first training samples or the plurality of second training samples into a training set and a test set. The training set may be used to train the model and the test set may be used to determine whether the training process has been completed. As a further example, the processing device 1206 may update the trained identification model and/or the second sub-model periodically or aperiodically based on one or more newly-generated training samples.

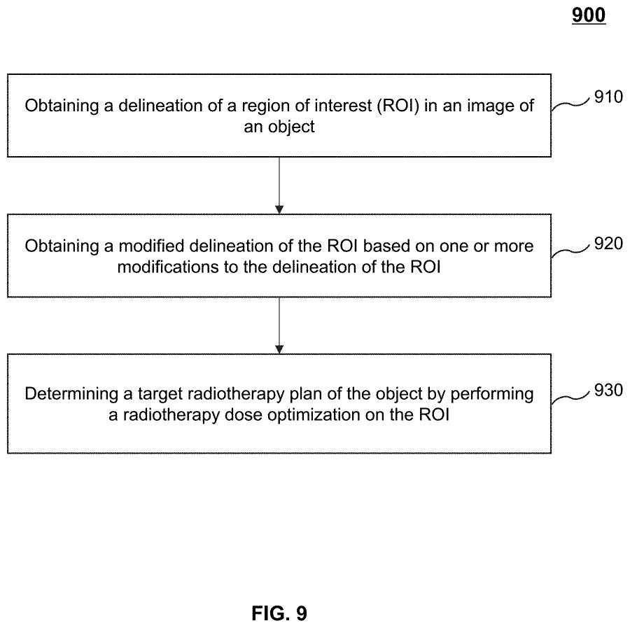

FIG. 9 is a flowchart illustrating an exemplary process for radiotherapy planning according to some embodiments of the present disclosure. In some embodiments, process 900 may be executed by the medical system 100. For example, the process 900 may be implemented as a set of instructions (e.g., an application) stored in a storage device (e.g., the storage 230). In some embodiments, the processing device 120A (e.g., the processor 220 of the computing device 200 and/or one or more modules illustrated in FIG. 3A) may execute the set of instructions and may accordingly be directed to perform the process 900.

In 910, the processing device 120A (e.g., the delineation module 320 illustrated in FIG. 3A, the processor 220 illustrated in FIG. 2) may obtain a delineation of a region of interest (ROI) in an image of an object.

In some embodiments, the image may include a CT image, a cone beam CT (CBCT) image, a fan beam CT (FBCT) image, an MR image, a PET-CT image, a PET-MR image, a SPECT-CT Image, a SPECT-MR Image, or the like, or a combination thereof. In some embodiments, the image may include the intermediate identification image and/or the identification image as described in connection with FIG. 4 and FIG. 7. In some embodiments, the ROI may include the at least one target region, the at least one OAR, and/or the specific region as described in connection with FIG. 4. In some embodiments, the ROI may further include a planned target volume (PTV).

In some embodiments, the delineation of the ROI may be previously determined and stored in a storage device (e.g., the storage 230, an external storage device). The processing device 120A may retrieve the delineation of the ROI from the storage device or the external system directly or via a network (e.g., the network 130). In some embodiments, the processing device 120A may obtain the delineation of the ROI. For example, the processing device 120A may obtain the delineation of the ROI using a model, for example, the trained identification model as described in connection with FIG. 4 and FIG. 7. In some embodiments, the processing device 120A may show the image of the object to a user (e.g., a radiation physicist or doctor) via a display (e.g., a liquid crystal display screen or an electronic ink display screen) of or in communication with the processing device 120A. The user may manually delineate the ROI in the image by an input device of the processing device 120A.

In 920, the processing device 120A (e.g., the delineation module 320 illustrated in FIG. 3A, the processor 220 illustrated in FIG. 2) may obtain a modified delineation of the ROI based on one or more modifications to the delineation of the ROI.

In some embodiments, the modified delineation of the ROI may be determined by manually modifying the delineation of the ROI. Specifically, after obtaining the delineation of the ROI, the processing device 120A may cause the delineation of the ROI to be shown to the user (e.g., a radiation physicist or doctor) via a display (e.g., a liquid crystal display screen or an electronic ink display screen) of or in communication with the processing device 120A. The user may manually modify the delineation of the ROI using an input device of the processing device 120A. Merely by way of example, the input device may be a touch layer covered on the display of or in communication with the processing device 120A, or a button, a trackball, or a touchpad set on a shell of the processing device 120A, or an external keyboard, trackpad, or mouse connected to the processing device 120A. The manual modification of the delineation of the ROI may improve the accuracy of the modified delineation of the ROI.

In 930, the processing device 120A (e.g., the determination module 330 illustrated in FIG. 3A, the processor 220 illustrated in FIG. 2) may determine a target radiotherapy plan of the object by performing a radiotherapy dose optimization on the ROI (also referred to as “optimizing a radiotherapy dose of the ROI”).

The radiotherapy dose optimization may refer to an iterative process, and the iterative process can be stopped at any time to generate a target radiotherapy plan.

The modifications of the delineation of the ROI and the radiotherapy dose optimization of the ROI may proceed in parallel or at least partially overlap temporally. As used herein, proceeding of two operations (e.g., modifications of the delineation of the ROI and the radiotherapy dose optimization of the ROI) at least partially overlapping temporally refers to that a time period during which the modifications of the delineation of the ROI occurs (e.g., performed manually by a user) or obtained overlaps at least in part with a time period during which the radiotherapy dose optimization of the ROI is performed. As used herein, proceeding of two operations (e.g., modifications of the delineation of the ROI and the radiotherapy dose optimization of the ROI) in parallel refers to that the operation of obtaining the modifications of the delineation of the ROI occurs (e.g., performed manually by a user) concurrently with the operation of optimizing the radiotherapy dose of the ROI. For example, while a user manually performs the modifications of the delineation of the ROI, the information of the modifications may be transferred to the processing device 120A, on the basis of which the processing device 120A may perform the operation of optimizing the radiotherapy dose of the ROI. Both operations may occur at the same time. Specifically, during the delineation of the ROI is modified, the processing device 120A may optimize, based on the modified portion of the delineation and the unmodified portion of the delineation of the ROI, the radiotherapy dose of the ROI to determine an intermediate radiotherapy plan of the object. Until the modifications of the delineation of the ROI is completed, the processing device 120A may determine the target radiotherapy plan of the object.

For example, a delineation of an ROI in one hundred image layers needs to be modified. When the delineation of the ROI in twenty image layers has been modified, the processing device 120A may optimize, based on the modified delineation of the ROI in the twenty image layers and unmodified delineation of the ROI in the remaining eighty image layers, the radiotherapy dose of the ROI to determine an intermediate radiotherapy plan of the object. When the delineation of the ROI in twenty-one image layers has been modified, the processing device 120A may optimize, based on the modified delineation of the ROI in the twenty-one image layers and unmodified delineation of the ROI in the remaining seventy-nine image layers, the radiotherapy dose of the ROI to determine another intermediate radiotherapy plan of the object. Until the modifications of the delineation of the ROI in the one hundred image layers is completed, the processing device 120A may optimize, based on the modified delineation of the ROI in the one hundred image layers, the radiotherapy dose of the ROI to determine the target radiotherapy plan of the object. In such cases, the radiotherapy dose optimization of the ROI is performed without waiting for the modifications of the delineation of the ROI in all image layers to be completed, so that the time for automatic or adaptive radiotherapy planning is reduced, thereby improving the efficiency of the automatic or adaptive radiotherapy planning.

In some embodiments, the target radiotherapy plan may be determined online during a radiotherapy treatment session of the object, which may reduce the count of trips for the patient to the hospital, shorten the overall time of the radiotherapy, and improve the efficiency of determining the target radiotherapy plan.

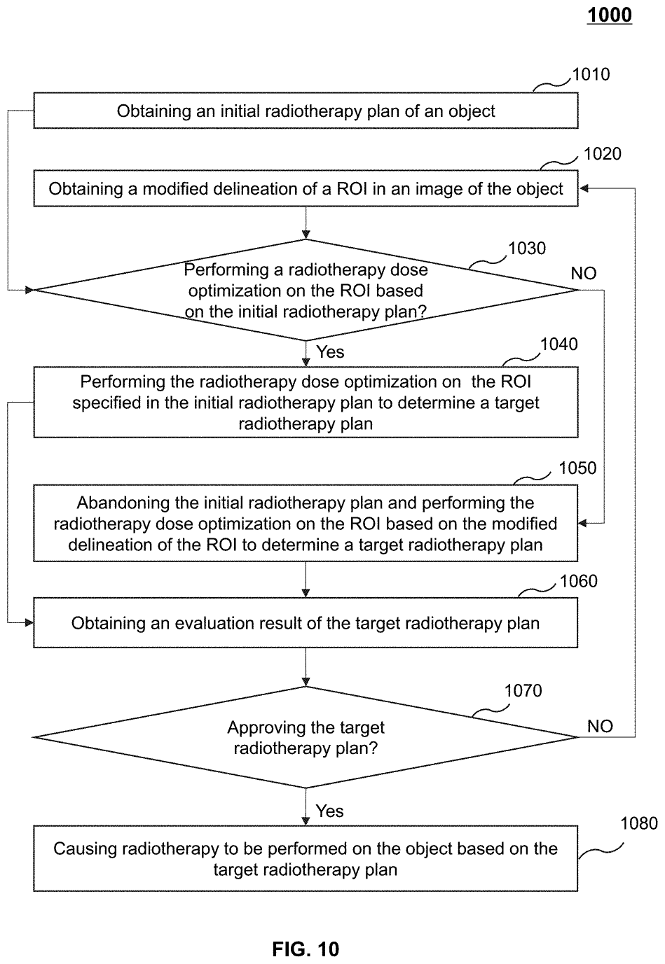

In some embodiments, the processing device 120A may optimize, based on a size (e.g., a volume) and/or a layer count of the ROI, the radiotherapy dose of the ROI to determine the target radiotherapy plan of the object. In some embodiments, the radiotherapy dose optimization of the ROI may include improving or reducing the radiotherapy dose of the ROI. More descriptions regarding the determination of the target radiotherapy plan may be found elsewhere in the present disclosure. See, e.g., FIGS. 10-12 and the descriptions thereof.

In some embodiments, while performing the radiotherapy dose optimization on the ROI, the processing device 120A may output in real time a dose distribution result and a dose volume histogram (DVH) corresponding to the radiotherapy dose being optimized. The user (e.g., a radiation physicist or doctor) may obtain, from the DVH, information related to the radiotherapy dose of the ROI, for example, a maximum radiotherapy dose in the ROI, a radiotherapy dose volume of the ROI, whether a dose index (e.g., D95) is up to a standard (e.g., a radiotherapy dose in a treatment prescription of the object), etc. In some embodiments, according to the dose distribution result and the DVH, the user may determine whether to adjust the modifications of the delineation of the ROI or limit a count of the OARs to adjust the target radiotherapy plan being determined. In some embodiments, according to the dose distribution result and the DVH, the user may formulate a further treatment prescription for the object. In such cases, the user may evaluate in real time the radiotherapy dose optimization result of the ROI, thereby improving the accuracy of the determined target radiotherapy plan.

It should be noted that the above description of the process 400 is merely provided for the purposes of illustration, and not intended to limit the scope of the present disclosure. For persons having ordinary skills in the art, multiple variations or modifications may be made under the teachings of the present disclosure. However, those variations and modifications do not depart from the scope of the present disclosure.

FIG. 10 is a flowchart illustrating an exemplary process for radiotherapy planning according to some embodiments of the present disclosure. In some embodiments, process 1000 may be executed by the medical system 100. For example, the process 1000 may be implemented as a set of instructions (e.g., an application) stored in a storage device (e.g., the storage 230). In some embodiments, the processing device 120A (e.g., the processor 220 of the computing device 200 and/or one or more modules illustrated in FIG. 3A) may execute the set of instructions and may accordingly be directed to perform the process 1000.

In 1010, the processing device 120A (e.g., the determination module 330 illustrated in FIG. 3A, the processor 220 illustrated in FIG. 2) may obtain an initial radiotherapy plan of an object.

In some embodiments, the initial radiotherapy plan of the object may be previously determined and stored in a storage device (e.g., the storage 230, an external storage device). The processing device 120A may retrieve the initial radiotherapy plan of the object from the storage device or the external system directly or via a network (e.g., the network 130). In some embodiments, the processing device 120A may determine the initial radiotherapy plan of the object. For example, the processing device 120A may obtain delineation of an ROI in an image (e.g., a CT image) of the object and determine the initial radiotherapy plan based on the delineation of the ROI. In some embodiments, the processing device 120A may obtain the delineation of the ROI using a model, for example, the trained identification model as described in connection FIG. 4 and FIG. 7. In some embodiments, the processing device 120A may cause the image of the object to be shown to a user (e.g., a radiation physicist or doctor) via a display (e.g., a liquid crystal display screen or an electronic ink display screen) of or in communication with the processing device 120A. The user may manually delineate the ROI in the image using an input device of the processing device 120A. Further, the processing device 120A may determine the initial radiotherapy plan of the object by optimizing (e.g., improving or reducing) the radiotherapy dose of the ROI based on the delineation of the ROI.

In 1020, the processing device 120A (e.g., the delineation module 320 illustrated in FIG. 3A, the processor 220 illustrated in FIG. 2) may obtain a modified delineation of the ROI. The obtaining of the modified delineation of the ROI may be performed in a similar manner as described in connection with operation 920, and the descriptions thereof are not repeated here.

In 1030, the processing device 120A (e.g., the determination module 330 illustrated in FIG. 3A, the processor 220 illustrated in FIG. 2) may determine, based on the modified delineation of the ROI, whether to perform the radiotherapy dose optimization on the ROI based on the initial radiotherapy plan.

The processing device 120A may determine whether the modified delineation of the ROI satisfies a condition. In some embodiments, the processing device 120A may determine whether a difference between parameters related to the modified delineation of the ROI and the delineation of the ROI is below a threshold. Merely by way of example, the parameters may include at least one of a volume, a layer count of the modified delineation of the ROI, etc. In response to that the difference between the parameters related to the modified delineation of the ROI and the delineation of the ROI is below the threshold (which indicates that the modifications of the delineation of the ROI is considered minor), the processing device 120A may determine that the modified delineation of the ROI satisfies the condition. In response to that the difference between the parameters related to the modified delineation of the ROI and the delineation of the ROI exceeds the threshold (which indicates that the modifications of the delineation of the ROI is considered substantial), the processing device 120A may determine that the modified delineation of the ROI fails to satisfy the condition.

In 1040, in response to that the modified delineation of the ROI satisfies the condition, the processing device 120A (e.g., the determination module 330 illustrated in FIG. 3A, the processor 220 illustrated in FIG. 2) may perform the radiotherapy dose optimization on the ROI specified in the initial radiotherapy plan to determine a target radiotherapy plan. In some embodiments, the processing device 120A may perform the radiotherapy dose optimization on the ROI by optimizing, using an automatic optimization algorithm, a shape, and a weigh of at least one segment of the initial radiotherapy plan. The automatic optimization algorithm may refer to an algorithm that automatically generates an optimization objective function and performs optimization iterations to generate a radiotherapy plan. Merely by way of example, the automatic optimization algorithm may include a script-based optimization algorithm, a historical data-based optimization algorithm, a predictive model-based optimization algorithm, or the like, or a combination thereof. A weight of a segment may refer to a ratio of monitor units (MUs) corresponding to the segment to MUs corresponding to a field that includes the segment. In such cases, when the modifications of the delineation of the ROI is considered minor, the target radiotherapy plan may be determined by performing the radiotherapy dose optimization on the ROI based on the initial radiotherapy plan, thereby improving the efficiency of the radiotherapy planning.

When the modified delineation of the ROI fails to satisfy the condition (which indicates that the modifications of the delineation of the ROI is considered substantial), it may become difficult or inappropriate to optimize the radiotherapy dose of the ROI based on the initial radiotherapy plan. Therefore, in 1050, in response to that the modified delineation of the ROI fails to satisfy the condition, the processing device 120A (e.g., the determination module 330 illustrated in FIG. 3A, the processor 220 illustrated in FIG. 2) may abandon the initial radiotherapy plan and perform the radiotherapy dose optimization on the ROI based on the modified delineation of the ROI to determine the target radiotherapy plan. In some embodiments, the processing device 120A may perform the radiotherapy dose optimization on the ROI by optimizing, based on the modified delineation of the ROI using an automatic optimization algorithm, a fluence map and/or at least one segment related to the radiotherapy dose of the ROI. In some embodiments, the processing device 120A may determine the target radiotherapy plan of the object based on the fluence map relating to the radiotherapy dose.

While optimizing the radiotherapy dose of the ROI, the processing device 120A may cause the obtained dose distribution result to be shown to the user via a display of or in communication with the processing device 120A. The user may review the dose distribution result and adjust the modifications of the delineation of the ROI based on the dose distribution result. As described in connection with operation 930, the modifications of the delineation of the ROI and the radiotherapy dose optimization of the ROI may be performed in parallel or at least partially overlap temporally. Therefore, operation 1020 and operations 1030-1050 may be performed in parallel or at least partially overlap temporally. For example, while performing operation 1020 to obtain the modified delineation of the ROI, the processing device 120A may perform operations 1030-1050 to determine the target radiotherapy plan, and while the modifications of the delineation of the ROI is completed, the target radiotherapy plan is obtained, thereby improving the efficiency of the radiotherapy planning.

In 1060, the processing device 120A (e.g., the determination module 330 illustrated in FIG. 3A, the processor 220 illustrated in FIG. 2) may obtain an evaluation result of the target radiotherapy plan. In some embodiments, the processing device 120A may cause the target radiotherapy plan to the user via a display of or in communication with the processing device 120A. The user may input an evaluation result of the target radiotherapy plan using an input device of or in communication with the processing device 120A.

In 1070, the processing device 120A (e.g., the determination module 330 illustrated in FIG. 3A, the processor 220 illustrated in FIG. 2) may determine whether to approve the target radiotherapy plan based on the evaluation result. In some embodiments, the evaluation result may include information related to whether to approve the target radiotherapy plan. The processing device 120A may determine, based on the evaluation result, whether to approve the target radiotherapy plan.

In 1080, if the target radiotherapy plan is approved, the processing device 120A (e.g., the determination module 330 illustrated in FIG. 3A, the processor 220 illustrated in FIG. 2) may cause radiotherapy to be performed on the object based on the target radiotherapy plan. If the target radiotherapy plan is not approved, the processing device 120A may perform operations 1020-1070 again.

It should be noted that the above description of the process 1000 is merely provided for the purposes of illustration, and not intended to limit the scope of the present disclosure. For persons having ordinary skills in the art, multiple variations or modifications may be made under the teachings of the present disclosure. However, those variations and modifications do not depart from the scope of the present disclosure.

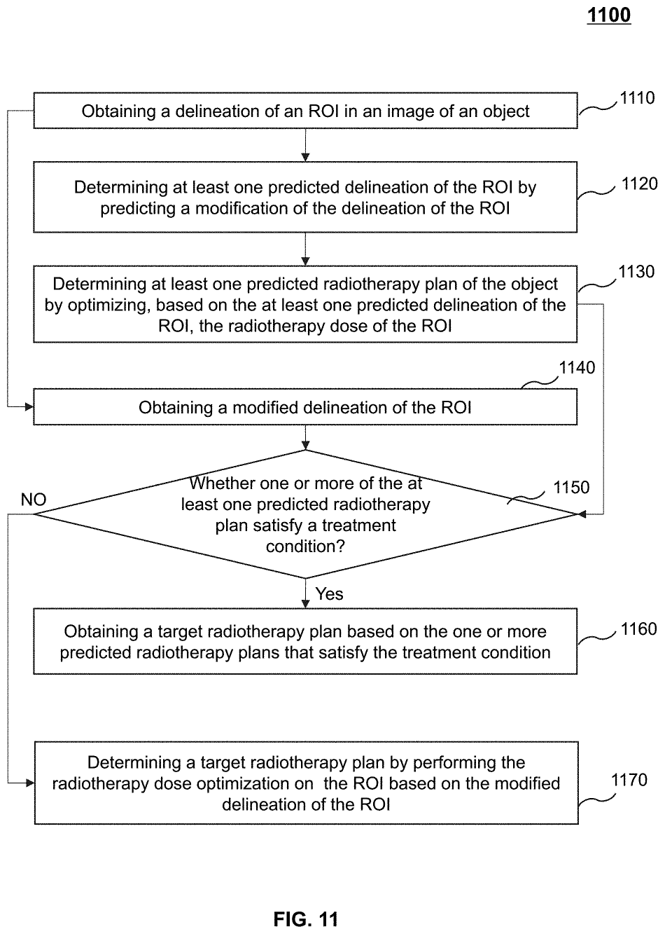

FIG. 11 is a flowchart illustrating an exemplary process for radiotherapy planning according to some embodiments of the present disclosure. In some embodiments, process 1100 may be executed by the medical system 100. For example, the process 1100 may be implemented as a set of instructions (e.g., an application) stored in a storage device (e.g., the storage 230). In some embodiments, the processing device 120A (e.g., the processor 220 of the computing device 200 and/or one or more modules illustrated in FIG. 3A) may execute the set of instructions and may accordingly be directed to perform the process 1100.

In 1110, the processing device 120A (e.g., the delineation module 320 illustrated in FIG. 3A, the processor 220 illustrated in FIG. 2) may obtain a delineation of an ROI in an image (e.g., a CT image) of an object. The obtaining of the delineation of the ROI may be performed in a similar manner as described in connection with operation 910, and the descriptions thereof are not repeated here.

In 1120, the processing device 120A (e.g., the delineation module 320 illustrated in FIG. 3A, the processor 220 illustrated in FIG. 2) may determine at least one predicted delineation of the ROI by predicting a modification of the delineation of the ROI.

In some embodiments, the processing device 120A may predict the modification of the delineation of the ROI using a trained machine learning model (e.g., a trained neural network model) that is different from the trained identification model as described in connection with FIG. 4 and FIG. 7. For example, the trained machine learning model and the trained identification model may have different structures, loss functions, or model parameters, but are obtained with the same training samples. In some embodiments, the processing device 120A may predict the modification of the delineation of the ROI by expanding or shrinking the delineation of the ROI within a certain range.

In 1130, the processing device 120A (e.g., the determination module 330 illustrated in FIG. 3A, the processor 220 illustrated in FIG. 2) may determine at least one predicted radiotherapy plan of the object by optimizing, based on the at least one predicted delineation of the ROI, the radiotherapy dose of the ROI.

In some embodiments, for each of the at least one predicted delineation of the ROI, the processing device 120A may optimize the radiotherapy dose of the ROI using a target function to determine a predicted radiotherapy plan corresponding to the predicted delineation of the ROI. Merely by way of example, the target function may be expressed as Equation 1 below:

$$min \{ {max}_{j} \{ {f}_{{objPTV}_{j}} ( {w}_{{PTV}_{j}} , \overline{D ( {x}_{j} )} ) \} \} , {x}_{j} ∈ {PTV}_{j} , \qquad ( 1 )$$

where PTVⱼ refers to a predicted delineation of the ROI, xⱼ refers to a voxel or pixel in the predicted delineation PTVⱼ, D(xⱼ) refers to a radiotherapy dose at the voxel or pixel xⱼ, and w_{PTV}_{ⱼ} refers to a weight of the predicted delineation PTVⱼ.

In 1140, the processing device 120A (e.g., the delineation module 320 illustrated in FIG. 3A, the processor 220 illustrated in FIG. 2) may obtain a modified delineation of the ROI. The obtaining of the modified delineation of the ROI may be performed in a similar manner as described in connection with operation 920, and the descriptions thereof are not repeated here.

In 1150, the processing device 120A (e.g., the determination module 330 illustrated in FIG. 3A, the processor 220 illustrated in FIG. 2) may determine, based on the modified delineation of the ROI, whether one or more of the at least one predicted radiotherapy plan satisfy a treatment condition (e.g., evaluating the at least one predicted radiotherapy plan).

In some embodiments, the treatment condition may be set according to a default setting of the medical system 100 or be adjustable under different situations. Merely by way of example, the treatment condition may include a preset radiotherapy dose or dose distribution. For example, for a predicted radiotherapy plan, the processing device 120A may determine whether a difference between a radiotherapy dose, in the predicted radiotherapy plan corresponding to a modified delineation of the ROI and the preset radiotherapy dose in the treatment condition exceeds a threshold. If the difference exceeds the threshold, the processing device 120A may determine that the predicted radiotherapy plan does not satisfy the treatment condition and perform operation 1170. If the difference does not exceed the threshold, the processing device 120A may determine that the predicted radiotherapy plan satisfies the treatment condition and perform operation 1160.

In 1160, the processing device 120A (e.g., the determination module 330 illustrated in FIG. 3A, the processor 220 illustrated in FIG. 2) may obtain a target radiotherapy plan based on the one or more predicted radiotherapy plans that satisfy the treatment condition.

For example, the processing device 120A may designate any one of the one or more predicted radiotherapy plans that satisfy the treatment condition as the target radiotherapy plan. As another example, the processing device 120A may show the one or more predicted radiotherapy plans that satisfy the treatment condition to a user (e.g., a radiation physicist or doctor) via a display (e.g., a liquid crystal display screen or an electronic ink display screen) of the processing device 120A. The user may manually select, via an input device of or in communication with the processing device 120A, one from the one or more predicted radiotherapy plans that satisfy the treatment condition as the target radiotherapy plan.

In 1170, the processing device 120A (e.g., the determination module 330 illustrated in FIG. 3A, the processor 220 illustrated in FIG. 2) may determine the target radiotherapy plan by performing the radiotherapy dose optimization on the ROI based on the modified delineation of the ROI. The determination of the target radiotherapy plan may be performed in a similar manner as described in connection with operation 930, and the descriptions thereof are not repeated here.

In the above embodiments, while performing operation 1140 to obtain the modified delineation of the ROI, the processing device 120A performs operations 1120-1130 to determine at least one predicted radiotherapy plan, which improves the efficiency of the radiotherapy planning, thereby reducing the time patients wait for treatment and improving patient experience. In addition, the target radiotherapy plan is determined by evaluating the at least one predicted radiotherapy plan, which may improve the accuracy of the determined target radiotherapy plan, thereby improving the treatment efficacy.

It should be noted that the above description of the process 1100 is merely provided for the purposes of illustration, and not intended to limit the scope of the present disclosure. For persons having ordinary skills in the art, multiple variations or modifications may be made under the teachings of the present disclosure. However, those variations and modifications do not depart from the scope of the present disclosure.

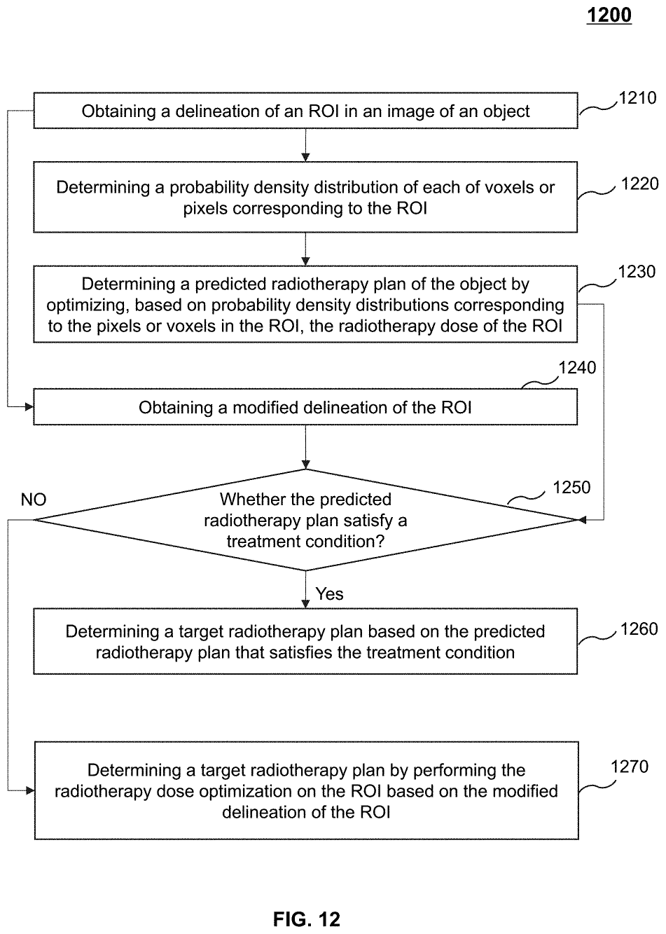

FIG. 12 is a flowchart illustrating an exemplary process for radiotherapy planning according to some embodiments of the present disclosure. In some embodiments, process 1200 may be executed by the medical system 100. For example, the process 1200 may be implemented as a set of instructions (e.g., an application) stored in a storage device (e.g., the storage 230). In some embodiments, the processing device 120A (e.g., the processor 220 of the computing device 200 and/or one or more modules illustrated in FIG. 3A) may execute the set of instructions and may accordingly be directed to perform the process 1200.

In 1210, the processing device 120A (e.g., the delineation module 320 illustrated in FIG. 3A, the processor 220 illustrated in FIG. 2) may obtain a delineation of an ROI in an image (e.g., a CT image) of an object. The obtaining of the delineation of the ROI may be performed in a similar manner as described in connection with operation 910, and the descriptions thereof are not repeated here.

In 1220, the processing device 120A (e.g., the delineation module 320 illustrated in FIG. 3A, the processor 220 illustrated in FIG. 2) may determine a probability density distribution of each of voxels or pixels corresponding to the ROI.

The probability density distribution may indicate a probability that the each voxel or pixel belongs to the ROI. If the delineation of the ROI is obtained using a model (e.g., the trained identification model as described in connection FIG. 4 and FIG. 7), an output of the model may include the probability density distribution of the each of voxels or pixels corresponding to the ROI. Then, the processing device 120A may determine the delineation of the ROI by converting, based on a probability threshold, the probability density distribution into a binary mask image.

In 1230, the processing device 120A (e.g., the determination module 330 illustrated in FIG. 3A, the processor 220 illustrated in FIG. 2) may determine a predicted radiotherapy plan of the object by optimizing, based on probability density distributions corresponding to the pixels or voxels in the ROI, the radiotherapy dose of the ROI.

In some embodiments, the processing device 120A may optimize the radiotherapy dose of the ROI using a target function to determine the predicted radiotherapy plan. Merely by way of example, the target function may be expressed as Equation 2 below:

min f_{objPVT}_{_{f}}(x(p),w_{PTV}_{_{f}},\vec{D(x)}), x∈PTV_{f} (2),

where PTV_{f} refers to the delineation of the ROI, x(p) refers to a probability density distribution of a voxel or pixel x corresponding to the ROI, D(x) refers to a radiotherapy dose at the voxel or pixel x, and w_{PTV}_{_{f}} refers to a weight of the delineation of the ROI.

In 1240, the processing device 120A (e.g., the delineation module 320 illustrated in FIG. 3A, the processor 220 illustrated in FIG. 2) may obtain a modified delineation of the ROI. The obtaining of the modified delineation of the ROI may be performed in a similar manner as described in connection with operation 920, and the descriptions thereof are not repeated here.

In 1250, the processing device 120A (e.g., the determination module 330 illustrated in FIG. 3A, the processor 220 illustrated in FIG. 2) may determine, based on the modified delineation of the ROI, whether the predicted radiotherapy plan satisfies a treatment condition (e.g., evaluating the predicted radiotherapy plan). The determination of whether the predicted radiotherapy plan satisfies the treatment condition may be performed in a similar manner as described in connection with operation 1150, and the descriptions thereof are not repeated here.

In some embodiments, the treatment condition may be set according to a default setting of the medical system 100 or be adjustable under different situations. Merely by way of example, the treatment condition may include a preset radiotherapy dose or dose distribution. For example, for a predicted radiotherapy plan, the processing device 120A may determine whether a difference between a radiotherapy dose, in the predicted radiotherapy plan corresponding to a modified delineation of the ROI and the preset radiotherapy dose in the treatment condition exceeds a threshold. If the difference exceeds the threshold, the processing device 120A may determine that the predicted radiotherapy plan does not satisfy the treatment condition and perform operation 1170. If the difference does not exceed the threshold, the processing device 120A may determine that the predicted radiotherapy plan satisfies the treatment condition and perform operation 1160.

In 1260, if the predicted radiotherapy plan satisfies the treatment condition, the processing device 120A (e.g., the determination module 330 illustrated in FIG. 3A, the processor 220 illustrated in FIG. 2) may obtain a target radiotherapy plan based on the predicted radiotherapy plan that satisfies the treatment condition. For example, the processing device 120A may designate the predicted radiotherapy plan that satisfies the treatment condition as the target radiotherapy plan.

In 1270, if the predicted radiotherapy plan does not satisfy the treatment condition, the processing device 120A (e.g., the determination module 330 illustrated in FIG. 3A, the processor 220 illustrated in FIG. 2) may determine the target radiotherapy plan by performing the radiotherapy dose optimization on the ROI based on the modified delineation of the ROI. The determination of the target radiotherapy plan may be performed in a similar manner as described in connection with operation 930, and the descriptions thereof are not repeated here.

In the above embodiments, while performing operation 1240 to obtain the modified delineation of the ROI, the processing device 120A performs operations 1220-1230 to determine the predicted radiotherapy plan, which improves the efficiency of the radiotherapy planning, thereby reducing the time patients wait for treatment and improving patient experience. In addition, the target radiotherapy plan is determined by evaluating the predicted radiotherapy plan, which may improve the accuracy of the determined target radiotherapy plan, thereby improving the treatment efficacy.

It should be noted that the above description of the process 1200 is merely provided for the purposes of illustration, and not intended to limit the scope of the present disclosure. For persons having ordinary skills in the art, multiple variations or modifications may be made under the teachings of the present disclosure. However, those variations and modifications do not depart from the scope of the present disclosure.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of this disclosure are not necessarily all referring to the same embodiment. Furthermore, the particular features, structures, or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.), or combining software and hardware implementation that may all generally be referred to herein as a “unit,” “module,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of a carrier wave. Such a propagated signal may take any of a variety of forms, including electromagnetic, optical, or the like, or any suitable combination thereof. A computer readable signal medium may be any computer readable medium that is not a computer readable storage medium and that may communicate, propagate, or transport a program for use by or in connection with an instruction-performing system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C#, VB. NET, Python or the like, conventional procedural programming languages, such as the “C” programming language, Visual Basic, Fortran 2103, Perl, COBOL 2102, PHP, ABAP, dynamic programming languages such as Python, Ruby, and Groovy, or other programming languages. The program code may execute entirely on the user's computer, partly on the user's computer, as a stand-alone software package, partly on the user's computer, and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed subject matter requires more features than are expressly recited in each claim. Rather, inventive embodiments lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

What is claimed is:

1. A system, comprising:
at least one storage device including a set of instructions; and
at least one processor in communication with the at least one storage device, wherein when executing the set of instructions, the at least one processor causes the system to perform operations including:
obtaining a delineation of a region of interest (ROI) in an image of an object, the ROI including at least one target region;
obtaining a modified delineation of the ROI based on one or more modifications to the delineation of the ROI; and
determining a target radiotherapy plan of the object by performing a radiotherapy dose optimization on the ROI, wherein
the modifications of the delineation of the ROI and the radiotherapy dose optimization of the ROI are performed at least partially overlap temporally.

2. The system of claim 1, wherein the modified delineation of the ROI is determined by manually modifying the delineation of the ROI.

3. The system of claim 1, wherein the operations further includes:
while performing the radiotherapy dose optimization on the ROI, outputting in real time a dose distribution result and a dose volume histogram (DVH) corresponding to the radiotherapy dose being optimized.

4. The system of claim 1, wherein the determining the target radiotherapy plan of the object includes:
determining, based on the modified delineation of the ROI, whether to perform the radiotherapy dose optimization on the ROI based on an initial radiotherapy plan.

5. The system of claim 4, wherein the determining, based on the modified delineation of the ROI, whether to perform the radiotherapy dose optimization on the ROI based on the initial radiotherapy plan includes:
in response to that the modified delineation of the ROI satisfies a condition, performing the radiotherapy dose optimization on the ROI based on the initial radiotherapy plan; and
in response to that the modified delineation of the ROI fails to satisfy the condition, abandoning the initial radiotherapy plan and performing the radiotherapy dose optimization on the ROI based on the modified delineation of the ROI.

6. The system of claim 5, wherein the operations further includes:
in response to that a difference between parameters related to the modified delineation of the ROI and the delineation of the ROI is less than a threshold, determining that the modified delineation of the ROI satisfies the condition, the parameters including at least one of a volume or a layer count of the modified delineation of the ROI.

7. The system of claim 5, wherein in response to that the modified delineation of the ROI satisfies the condition, the performing the radiotherapy dose optimization on the ROI based on the initial radiotherapy plan includes:
performing the radiotherapy dose optimization on the ROI by optimizing, using an automatic optimization algorithm, a shape and a weight of at least one segment of the initial radiotherapy plan.

8. The system of claim 5, wherein in response to that the modified delineation of the ROI fails to satisfy the condition, the performing the radiotherapy dose optimization on the ROI based on the modified delineation of the ROI includes:
performing the radiotherapy dose optimization on the ROI by optimizing, based on the modified delineation of the ROI using an automatic optimization algorithm, a fluence map related to the radiotherapy dose of the ROI.

9. The system of claim 8, wherein the determining the target radiotherapy plan of the object includes:
determining the target radiotherapy plan of the object based on the fluence map relating to the radiotherapy dose.

10. The system of claim 1, wherein the determining the target radiotherapy plan of the object includes:
determining at least one predicted delineation of the ROI by predicting a modification of the delineation of the ROI;
determining at least one predicted radiotherapy plan of the object by performing, based on the at least one predicted delineation of the ROI, the radiotherapy dose optimization on the ROI; and
determining the target radiotherapy plan of the object by evaluating, based on the modified delineation of the ROI, the at least one predicted radiotherapy plan.

11. The system of claim 1, wherein the determining the target radiotherapy plan of the object includes:
determining a probability density distribution of each of voxels or pixels corresponding to the ROI, the probability density distribution indicating a probability that the each voxel or pixel belongs to the ROI;
determining a predicted radiotherapy plan of the object by performing, based on probability density distributions corresponding to the pixels or voxels in the ROI, the radiotherapy dose optimization on the ROI; and
determining the target radiotherapy plan of the object by evaluating, based on the modified delineation, the predicted radiotherapy plan.

12. The system of claim 1, wherein the target radiotherapy plan is determined online during a radiotherapy treatment session of the object that includes a delivery of the radiotherapy dose to the at least one target region of the object.

13. The system of claim 1, wherein:
the image of the object is an identification image that is determined using a trained identification model, the trained identification model being configured to delineate the at least one target region and the specific region in an initial image to determine the identification image, and
the operations further include determining a radiotherapy dose of the specific region based on the identification image.

14. The system of claim 13, wherein
the trained identification model includes a first sub-model and a second sub-model, and
the determining the identification image using the trained identification model includes:
determining, using the first sub-model, an intermediate identification image based on the initial image, the intermediate identification image including delineations of the at least one target region; and
determining the identification image by delineating the specific region in the intermediate identification image using the second sub-model.

15. The system of claim 13, wherein the determining the radiotherapy dose of the specific region based on the identification image includes:
determining an optimization objective by performing a dose prediction based on the delineation of the specific region;
obtaining a dose constraint corresponding to the specific region based on the optimization objective; and
determining the radiotherapy dose of the specific region based on the dose constraint.

16. The system of claim 15, wherein the obtaining the dose constraint corresponding to the specific region based on the feature of the specific region includes:
determining, using a dose distribution prediction model, the dose constraint corresponding to the specific region.

17. The system of claim 16, wherein the second sub-model is obtained by a training process including:
obtaining a plurality of training samples, each of the plurality of training samples including a sample image and a label image corresponding to the sample image, the sample image including delineations of at least one sample target region, the label image including a delineation of a sample specific region; and
obtaining the second sub-model by training, based on the plurality of training samples, a preliminary second sub-model.

18. A system, comprising:
at least one storage device including a set of instructions; and
at least one processor in communication with the at least one storage device, wherein when executing the set of instructions, the at least one processor causes the system to perform operations including:
obtaining an initial image of an object, the initial image including at least one target region;
determining an identification image using a trained identification model, the trained identification model being configured to delineate the at least one target region and a specific region in the initial image to determine the identification image; and
determining a radiotherapy dose of the specific region based on the identification image.

19. The system of claim 18, wherein
the trained identification model includes a first sub-model and a second sub-model, and
the determining the identification image using the trained identification model includes:
determining, using the first sub-model, an intermediate identification image based on the initial image, the intermediate identification image including delineations of the at least one target region; and
determining the identification image by delineating the specific region in the intermediate identification image using the second sub-model.

20. A method for radiotherapy planning, implemented on a computing device having at least one storage device storing a set of instructions, and at least one processor in communication with the at least one storage device, the method comprising:
obtaining a delineation of a region of interest (ROI) in an image of an object, the ROI including at least one target region;
obtaining a modified delineation of the ROI based on one or more modifications to the delineation of the ROI; and
determining a target radiotherapy plan of the object by performing a radiotherapy dose optimization on the ROI, wherein
the modifications of the delineation of the ROI and the radiotherapy dose optimization of the ROI are performed at least partially overlap temporally.
